(19) United States
(12) Patent Application Publication
(10) Pub. No.: US 2006/0111449 A1
(43) Pub. Date: May 25, 2006

(54) Use of long-chain alcohols for effectively lowering elevated cholesterol levels

(75) Inventors: Bernhard Kammermeier, Muenchen (DE); Franz-Leo Heinrichs, Gablingen (DE); Ernst Krendlinger, Friedberg (DE); Barbara Danner, Althegnenberg (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/283,209

(22) Filed: Nov. 18, 2005

(30) Foreign Application Priority Data
Nov. 19, 2004 (DE) 10 2004 055 858.2

(51) Int. Cl. A61K 31/045

(52) U.S. Cl. 514/724

(57) ABSTRACT

The invention relates to the use of long-chain alcohols for effectively lowering elevated cholesterol levels, wherein the long-chain alcohols are obtained from montan wax alcohols and Guerbet alcohols and their beeswax analogs.

The present invention is described in the German priority application No. 102004055858.2, filed Nov. 19, 2004, which is hereby incorporated by reference as is fully disclosed herein.

The invention relates to the use of long-chain alcohols for effectively lowering elevated cholesterol levels.

The cholesterol level can, for instance, be defined as follows: The HDL cholesterol (high density lipoprotein cholesterol) is also termed the “good” cholesterol since it appears to protect the blood vessels from fat deposits. When assessing the cholesterol level, it is necessary to distinguish between the “good” cholesterol and the “bad” cholesterol. Cholesterol is mainly present in the blood in two different transport forms. The “good” HDL transports the cholesterol from the blood vessels to the liver. In this way, it protects the blood vessels and can prevent arteriosclerosis. It would consequently be desirable to have a high concentration of HDL in the blood.

By contrast, the “bad” LDL (low density lipoprotein cholesterol) transports the cholesterol from the periphery to the cells. When there is a surplus, the LDL can release cholesterol, which then becomes deposited on the blood vessel walls and can lead to arteriosclerosis. The ratio of LDL to HDL in the blood should consequently be as low as possible. Ideally, the aim should be to achieve an LDL level of <130 milligrams per deciliter and an HDL level of >45 milligrams per deciliter.

It is known that, at a dose of from 10 to 20 mg/day, aliphatic long-chain alcohols derived from the wax of Saccharum officinarum (policosanol) are able to lower the total cholesterol level by from 17 to 21% and to lower the LDL level by from 21 to 29%, in connection with which the HDL values additionally rise by from 8 to 15% (E. Ernst, MMW No. 23/2002, page 20).

The empirical formula of the abovementioned wax alcohols is H₃C—(CH₂)ₙ—CH₂—OH, where the chain length n varies from 24 to 32 carbon atoms. In this connection, investigations carried out in animal experiments have shown that, in addition to the abovementioned effects, this substance mixture exhibits further antiatherogenic effects and other positive effects from the health point of view, with no adverse effects having been found. In the three-year comparison, comparative studies demonstrate an effect which is equivalent to that of conventional statins (simvastatin and pravastatin were compared) with the effect being exhibited in the absence of a side-effect profile.

However, a disadvantage of the aliphatic long-chain alcohols from the Saccharum officinarum wax are the natural variations in product quality, in the alcohol composition (chain-length distribution) and in other quality-determining features which arise as a result of natural causes in substances which are obtained from renewable raw materials.

Furthermore, the abovementioned policosanols can only be obtained from the corresponding natural raw material in very small quantities (a few percent) after employing elaborate physicochemical isolation and purification methods.

The products can only with very great difficulty be prepared free from the byproducts which always appear in such complex mixtures derived from natural sources.

In particular, however, microbial contaminations (fungi and their mycotoxins, bacteria, protozoa and viral components) resulting from natural environmental conditions, or else pesticides and toxins of anthropogenic origin, can also be present.

Thus far, there has been a lack of suitable long-chain unbranched alcohols (and, where appropriate, branched alcohols which are relevant for physiological investigations) which can be made available for lowering elevated cholesterol levels.

In addition, it would be desirable for the products which are to be employed not to exhibit any microbial contaminations arising from natural environmental conditions and not to exhibit any pesticides or toxins of anthropogenic origin, either.

The invention therefore relates to the use of long-chain alcohols for effectively lowering elevated cholesterol levels wherein the long-chain alcohols are obtained from montan wax alcohols and Guerbet alcohols and their beeswax analogs.

The long-chain alcohols are preferably obtained from the waxes by means of saponification reactions.

The long-chain alcohols are preferably also obtained from montan wax by means of oxidative cleavage.

The long-chain alcohols preferably do not exhibit any microbial contaminations.

The long-chain alcohols preferably do not exhibit any contaminations with pesticides or toxins, either.

Advantageously, the long-chain unbranched alcohols which are to be derived from the montan waxes and also the Guerbet alcohols as well as their beeswax analogs exhibit a structural composition which is analogous to that of the policosanols which were mentioned at the outset and therefore exhibit effects with regard to influencing lipid metabolism which are similar to, and in some cases even more powerful than, those of the policosanols.

As long-chain unbranched alcohols, said montan wax alcohols exhibit a gas-chromatographic fingerprint which is highly similar to that of the policosanols mentioned at the outset and were also acknowledged to be nontoxic in toxicological studies on tissues.

The great advantage of the montan wax alcohols, and consequently the technical innovation, lies, in the first place, in their ready, and quantitatively virtually “unlimited”, availability and accessibility from montan waxes and in the harmlessness, with regard to tissue toxicity, of these raw materials and their derivatives.

Furthermore, the technique of isolating said waxes on an industrial scale qualitatively and in a reproducible manner is well established and the chemistry required for releasing the alcohols is a well-known basic reaction which is safe both technically and from the point of view of environmental toxicity.

For example, the required long-chain alcohols can be cleaved from the wax by means of saponification reactions, and then extracted in a purifying manner, using the well-known industrial methods.

The montan wax can also be cleaved oxidatively by means of an industrially available chromosulfuric acid oxidation and the resulting montan wax acids can be converted reductively into the required long-chain alcohols.

The relatively large natural variations in product quality which occur as a result of natural causes in substances derived from renewable raw materials (for instance in the case of the policosanols) are not to be expected in the case of said long-chain alcohols which can be obtained using these large-scale industrial processes.

Guerbet alcohols can also be obtained from readily accessible raw materials using a very simple chemical reaction.

With their additional β-branching and better control/adjustment of the total chain length, these long-chain alcohols highlight another possibility of influencing lipid metabolism in the above-mentioned sense.

The fact that the metabolic utilizability of these alcohols, with their additional β-branching, is poorer than that of unbranched analogs, because of the branching, results, as in the case of the montan wax alcohols, in there being only a slight possibility, or no possibility, of metabolic energy generation (“fat replacers” in literature: “Natural and Synthetic Substances Related to Human Health”, Pure and Appl. Chem. 2002, pages 1976-1977).

Both montan wax alcohols and Guerbet alcohols can be synthesized in a tailor-made manner to a required chain length distribution (which is not the case with the abovementioned policosanols) and can be prepared free of the byproducts which always appear in complex mixtures derived from natural sources.

In particular, no microbial contaminations (fungi and their mycotoxins, bacteria, protozoa and viral components) arising from natural environmental conditions, or else pesticides and toxins of anthropogenic origin, are to be expected, either.

In contrast to the policosanols mentioned at the outset, the product yield for montan wax alcohols from montan waxes is theoretically 100% (after additional reduction of the acid component in the montan wax) whereas, after employing elaborate physicochemical isolation and purification methods, the policosanols are at best obtained from the natural raw material in the single-figure percent range.

The product content of montan wax alcohols or Guerbet alcohols can be determined using a simple and well established analysis without the necessity of any microbiological product control.

Another advantage is a secure raw material basis with constant product quality and yield quantity without the natural variations to which a natural substance is usually subject.

A further advantage of the present invention is to be seen in the fact that, because of the very similar molecular distributions and structures, only dose quantities of montan wax alcohols or Guerbet alcohols (or else their beeswax analogs) which are analogous to (or less than) those of the policosanols are required for achieving the cholesterol-lowering effect while retaining the oral administration form.

Nor, because of the lack of possibilities for metabolism, do any additional caloric loadings arise when employing long-chain montan wax alcohols or Guerbet alcohols (“Natural and Synthetic Substances Related to Human Health”, Pure and Appl. Chem. 2002, pages 1957-1985).

1. A method for effectively lowering elevated cholesterol levels, comprising the step of administering an effective amount of long-chain alcohols obtained from montan wax alcohols, Guerbet alcohols or beeswax analogs thereof.

2. The use as claimed in claim 1, wherein the long-chain alcohols are obtained from the waxes by of saponification reactions.

3. The use as claimed in claim 1, wherein the long-chain alcohols are obtained from montan wax by oxidative cleavage.

4. The use as claimed in claim 1, wherein the long-chain alcohols do not exhibit any microbial contaminations.

5. The use as claimed in claim 1, wherein the long-chain alcohols do not exhibit any contaminations with pesticides or toxins.

6. A composition for lowering elevated cholesterol levels, comprising long-chain alcohols obtained from montan wax alcohols, Guerbet alcohols or beeswax analogs thereof.

7. A composition for lowering elevated cholesterol levels, comprising at least one of a long-chain alcohol obtained from montan wax alcohols, Guerbet alcohols or beeswax analogs thereof.

8. A method for lowering elevated cholesterol levels, comprising the step of administering an effective amount of at least one of a long-chain alcohol obtained from montan wax alcohols, Guerbet alcohols or beeswax analogs thereof.
